(19) United States
(12) Patent Application Publication
(10) Pub. No.: US 2015/0216708 A1
(43) Pub. Date: Aug. 6, 2015

(54) Adjustable Collar

(71) Applicant: Aspen Medical Partners, LLC, Irvine, CA (US)

(72) Inventors: Geoffrey Garth, Long Beach, CA (US); Steven Burke, Huntington Beach, CA (US)

(21) Appl. No.: 14/638,366

(22) Filed: Mar. 4, 2015

Related U.S. Application Data
(60) Provisional application No. 61/947,676, filed on Mar. 4, 2014.

(51) Int. Cl. A61F 5/055

(52) U.S. Cl. CPC A61F 5/055; A61F 2250/0004

(57) ABSTRACT

Adjustable collars providing improved levels of adjustability and ease of use are provided. Some adjustable collars can comprise a chin piece coupled to a collar body via a chin support that is pivotally and slidably coupled with the collar body and allows for an adjustment of a position of the chin piece relative to the collar body.

This application claims priority to U.S. provisional patent application No. 61/947676 filed Mar. 4, 2014. This and all other extrinsic materials discussed herein are incorporated by reference in their entirety.

### FIELD OF THE INVENTION

The field of the invention is cervical collars, and more specifically adjustable cervical collars.

### BACKGROUND

The following description includes information that may be useful in understanding the present invention. It is not an admission that any of the information provided herein is prior art or relevant to the presently claimed invention, or that any publication specifically or implicitly referenced is prior art.

Cervical collars are often used to maintain a spine in neutral alignment by supporting a user's chin at a particular position. Because users of the collars are sized and proportioned differently and making collars in different sizes typically leads to tremendous stocking and tracking burdens, many have attempted to provide one-size-fits-most adjustable collars that can be used by different users having different or varying heights, weights and proportions.

Some examples of known adjustable collars are described in U.S. Pat. No. 6,663,581 to Calabrese and 8,449,485 to Modglin; U.S. Pat. App. Pub. No. 2012/0209160 to Thorgilsdottir et al.; and WIPO Publ. No. 2013/036368 to Modglin. These and all other publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Unfortunately, known adjustable collars suffer various shortcomings. For example, known adjustable collars generally fail to provide an optimal or desired level of adjustability and ease of use. Thus, there is still a need for improved adjustable collars.

### SUMMARY OF THE INVENTION

The inventive subject matter provides apparatus, systems and methods in which an adjustable collar comprises a chin piece coupled to a collar body via a chin support. In some aspects, the collar further includes an adjustment mechanism that allows for an adjustment of the chin piece relative to the collar body. The adjustment mechanism preferably allows for at least bidirectional adjustment of the chin piece relative to the collar body. In some preferred embodiments, the chin piece could be temporarily fixed along one of a plurality of positions relative to the collar body as the adjustment mechanism is adjusted. An exemplary adjustment mechanism of the inventive subject matter could comprise (i) two movable adjustment members disposed on opposite end portions of a chin support, each adjustment member being coupled to or including a projection, and (ii) two recesses or slots disposed on a collar body, aligning with the two projections and comprising a plurality of levels or locking positions. The slots can each advantageously be sized and dimensioned to receive a corresponding projection that can move along the length and levels of the slot.

It is contemplated that a chin support could be coupled to a collar body in any suitable manner. For example, the chin support could be pivotally, rotatably or slidably coupled with the collar body at one or more attachment points (e.g., 1, 2, 3, 4, 5, etc.). In some embodiments, the chin support is pivotally attached to the collar body at two points (e.g., left rear portion and right rear portion, etc.), and slidably coupled to the collar body at two additional points (e.g., via notched rails of the collar body and projection of adjustment mechanism disposed on left and right front portions, etc.). Where the projections are moved from a lower level to a higher level, the chin support pivots at the pivot attachment points and adjusts a position of the chin support relative to the collar body. The chin support could be coupled with a chin piece, wherein an adjustment of the adjustment mechanism would also adjust a position of the chin piece relative to the collar body.

In some embodiments the projections of adjustment mechanisms could be disposed on or otherwise coupled to one or more adjustment members. An adjustment member could comprise a finger wall or stop (e.g., 380 of FIG. 3C), and be sufficiently flexible or movable to allow a wearer to reduce a distance between the adjustment members by at least lcm, and more preferably by at least 2 cm. The adjustment mechanism could be disposed on any suitable collar component or components (e.g., chin support, collar body, chin piece, etc.). In some preferred embodiments, the adjustment mechanism comprises adjustment members having finger stops disposed on left and right sides of a chin support, and a distance between the finger stops is less than or equal to 6 inches, and more preferably less than or equal to 5 inches, when the adjustment members are at a resting position. Additionally or alternatively, retractable members, notched rails, slots or any other portions of an adjustment mechanism will be substantially horizontally aligned when the collar is worn by a wearer.

Viewed from another perspective, the inventive subject matter includes an adjustable collar for use around a neck of a wearer comprising a chin piece coupled to a collar body via a chin support. The adjustable collar could further comprise an adjustment mechanism comprising one or more retractable members including or coupled with one or more projections, preferably in a 1:1 ratio, and one or more notched or recessed rails, each notched rail including a plurality of levels (at the notches/recesses). The retractable members and notched rail could be disposed on a chin support, collar body, or a combination thereof. For example, a chin support could comprise the notched rails and a collar body could comprise the retractable members and projections. Additionally or alternatively, the chin support could comprise the retractable members and projections and the collar body could comprise the notched rails. The distance between the projections could be the same as a distance between at least portions of the slots such that the chin support is positioned partially or completely behind or in front of the collar body when the pieces are coupled together.

In some aspects of the inventive subject matter, some notches of an adjustment mechanism could be tapered in a manner that allows for movement of corresponding projection(s) in a preferred direction. For example, an angle between a back wall of a recess and a top wall of the recess (e.g., >90 degrees) could be greater than an angle between a back wall of the recess and a bottom wall of the recess (e.g., approximately 90 degrees) such that a corresponding projection would more easily move to a higher notch than a lower notch.

The inventive subject matter also provides alternative embodiments of apparatus, methods and systems in which a collar comprises a chin piece coupled to a collar body, and one or more adjustment mechanisms for adjusting a position of the chin piece relative to the collar body. It should be appreciated that the chin piece could be directly or indirectly coupled to the collar body in any commercially suitable manner.

Some adjustment mechanisms of the inventive subject matter could comprise first and second slots that are disposed on a collar body and sized and dimensioned to receive portions (e.g., end portions, etc.) of a chin support such that the first and second slots act as guides or rails when a vertical position of the chin support, optionally comprising a chin piece, is adjusted relative to the collar body.

In other aspects of the inventive subject matter, an adjustment mechanism could comprise first and second looped lines and a rotatable member. In such embodiments, the looped lines could extend between the collar body and the chin piece, and the collar body could be coupled to the rotatable member (e.g., the rotatable member could be disposed through an slot of the collar body, etc.). The looped lines could mate with the rotatable member such that a rotation of a knob portion of the rotatable member in a first direction causes the looped lines to wind around a spool portion of the rotatable member. Additionally or alternatively, the looped lines could mate with the rotatable member such that a rotation of the knob portion of the rotatable member in a second direction (e.g., a direction opposite the first direction, etc.) causes the looped lines to unwound from the spool portion of the rotatable member.

In yet other aspects of the inventive subject matter, an adjustment mechanism could comprise a single looped line and a rotational member that includes a knob and a stool. The looped line could extend from the collar body to first and second chin support members (e.g., right and left chin support members, etc.) that support a chin piece. The looped line could mate with the spool such that a rotation of the knob in a first direction causes the line to wind around the spool, and a rotation of the knob in a second (e.g., opposite, etc.) direction causes the line to unwind from the spool. It can generally be preferred that the line(s) are of sufficient sturdiness to at least one of (1) extend into a slot of the inventive subject matter, and (2) support a chin of a wearer.

In yet further aspects of the inventive subject matter, an adjustment mechanism could comprise a rotatable member having threads cut in first and second different directions from a mid-point. A first rod could include threads complementary to the threads cut in the first direction, and a second rod could include threads complementary to the threads cut in the second direction. The first and second rods could be removably attached to the rotatable member at opposing ends, and couple with a chin piece or chin support at opposing ends such that a rotation of the rotatable member in a first direction shortens a distance between a chin piece/support and the rotatable member, and a rotation of the rotatable member in a second direction increases a distance between the chin piece/support and the rotatable member.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1A is a perspective view of a collar of the inventive subject matter in a collapsed configuration.

FIG. 1B is a perspective view of the collar of FIG. 1A in an expanded configuration.

FIG. 2 is a perspective view of a collar of the inventive subject matter with an adjustment mechanism detached from a collar body.

FIG. 3A is a front view of a collar of the inventive subject matter in a collapsed configuration.

FIG. 3B is a front view of the collar of FIG. 3A in an expanded configuration.

FIG. 3C is a perspective view of the collar of FIGS. 3A and 3B in an expanded configuration.

FIG. 4 is a front view of an alternative adjustable collar of the inventive subject matter.

FIG. 5 is a front view of an alternative adjustable collar of the inventive subject matter.

FIG. 6 is a perspective view of an alternative adjustment mechanism of the inventive subject matter.

FIG. 7 illustrates a portion of an alternative adjustable collar having an adjustment mechanism.

FIG. 8 illustrates a collar of the inventive subject matter where a chin piece is directly coupled with a collar body.

FIG. 9 is a front view of an alternative adjustable collar of the inventive subject matter having flexible threaded rods.

FIG. 10 is a front view of an alternative adjustable collar of the inventive subject matter.

FIG. 11A is a side perspective view of an alternative adjustable collar of the inventive subject matter having one or more locking tabs.

FIG. 11B illustrates a portion of a collar of the inventive subject matter having a plurality of bumps.

FIG. 11C illustrates an interlocking portion of a collar of the inventive subject matter composed of a surface of a chin piece and an abutting surface of a collar body.

### DETAILED DESCRIPTION

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

While the discussion herein is directed to cervical collars, one should appreciate that the adjustment mechanisms provided herein could be used with any suitable orthotic device having at least two components, or a portion thereof.

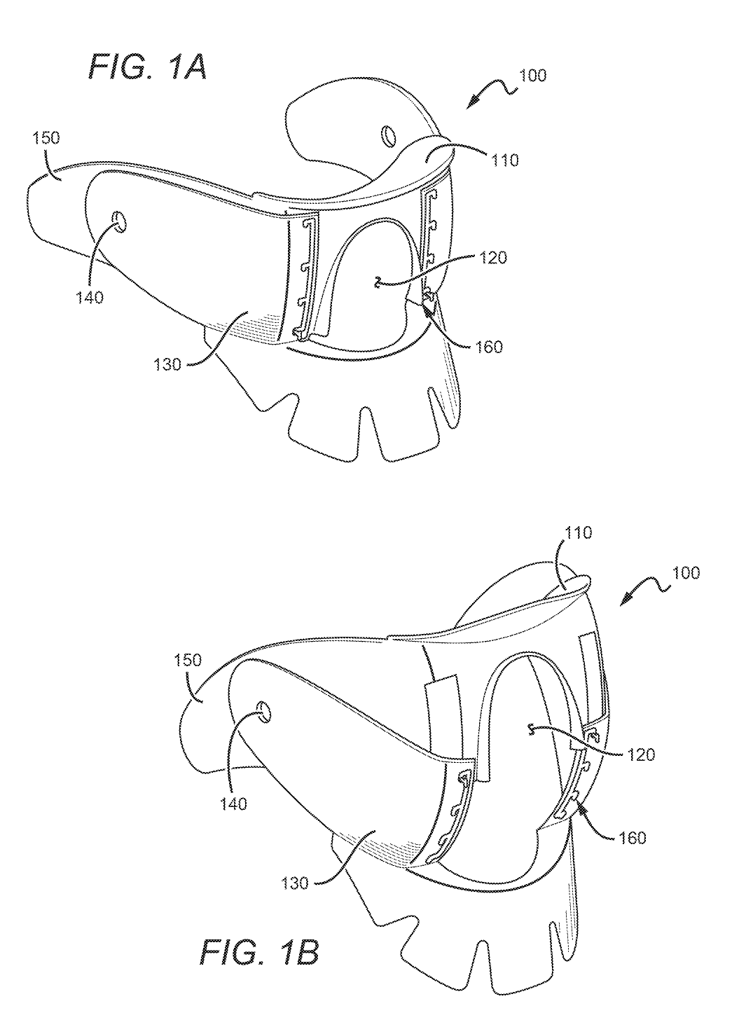

FIGS. 1A-1B are perspective views of an adjustable collar of the inventive subject matter in a collapsed configuration and an expanded configuration, respectively. Collar 100 comprises a chin piece 110 attached to a chin support 150, which is pivotally coupled to a collar body 130 at attachment point 140 (among other attachment points). The collar 100 further comprises an adjustment mechanism 160 including first and second projections (not shown) disposed on chin support 150, which slide along first and second slots or tracks of collar body 130 and allows for an adjustment of a position of chin piece 110 relative to collar body 130. As illustrated, the chin support 150 and collar body 130 are sized and dimensioned to provide a tracheal aperture 120 that can vary in size and allows for access to a frontal portion of the neck or throat of a wearer.

It is contemplated that adjustment mechanism 160 could comprise a notched recess or rail in addition to, or alternatively to, one or more slots or tracks. Each notch of the notched recess or rail could represent a different level or adjustment point, and adjustment of a projection from a first notch to a second different notch adjusts a vertical position of a chin piece relative to the collar body. It is also contemplated that in some embodiments the chin support could comprise one or more apertures or notched recesses, and that the collar body could comprise one or more projections. The projections could be positioned on a front surface or a back surface of at least one of the chin support and collar body. Where the projections are on a back surface of a collar component (e.g., the chin support, etc.), the corresponding component (e.g., the collar body, etc.) could comprise a notched recess on a front surface. Where the projections are positioned on a front surface of the collar component, the corresponding component could comprise a notched recess on a back surface.

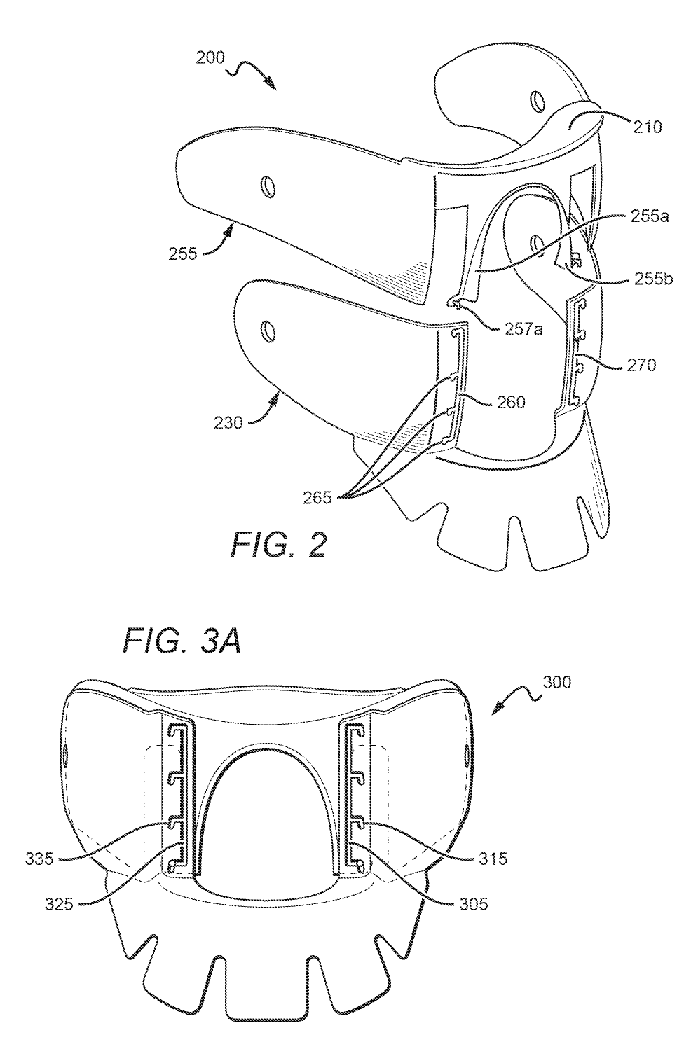

FIG. 2 is a perspective view of another adjustable collar 200 of the inventive subject matter wherein the chin support 255 and chin piece 210 are removed from the collar body 230 for illustrative purposes. Chin support 255 comprises chin piece 210, and further includes first adjustment member 255a and second adjustment member 255b, which can advantageously be biased away from a tracheal aperture, and retracted towards the tracheal aperture by a user for adjustment of a position of the chin piece 210 relative to the collar body 230. Collar body 230 comprises a first slot (aperture) or rail (recess) 260 and a second slot or rail 270, each of which include a plurality of levels (e.g., 265, etc.) optionally having curved ends where a projection can rest. First projection 257a is sized and dimensioned such that at least a stem or mid-portion of first projection 257a can be received by first aperture 260 and moved from one level to another via a retraction or other movement of first adjustment member 255a. Similarly, a second projection (not shown) can be sized and dimensioned such that at least a portion of the second projection can be received by second slot or rail 270 and moved from one level to another via a retraction or other movement of the second adjustment member. It should be appreciated that the first adjustment member, second adjustment member, first projection, second projection, first slot or rail and second slot or rail could all be considered a part of an adjustment mechanism of adjustable collar 200. In some embodiments, the adjustment mechanism could include one or more finger stops that assist a user in retracting first and second adjustment members.

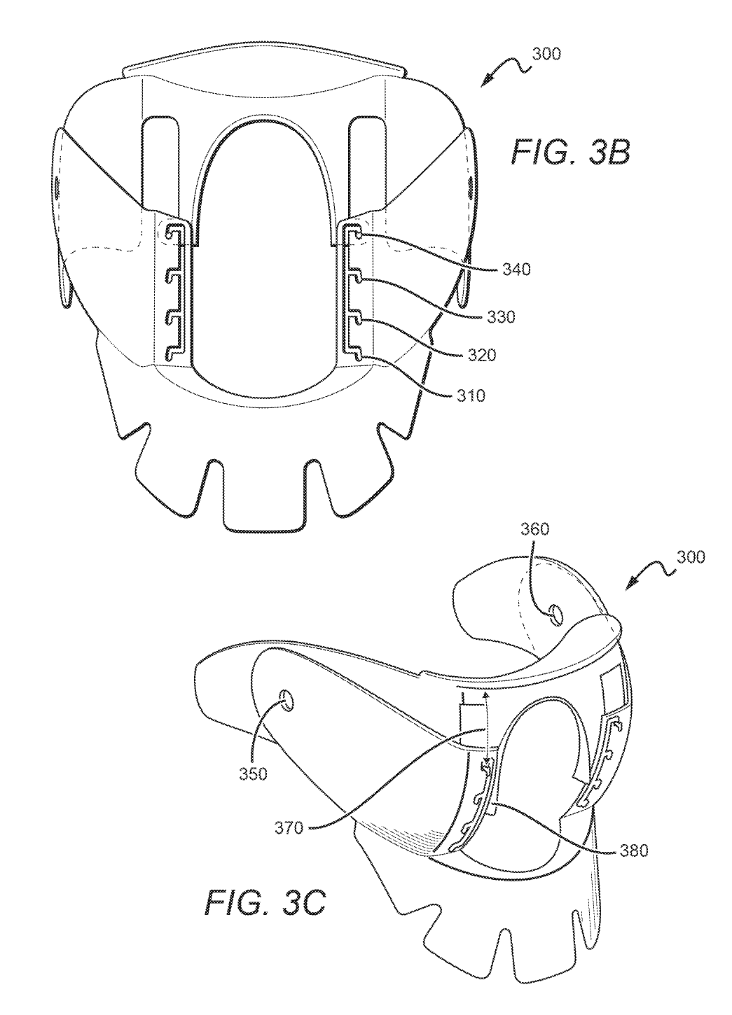

FIG. 3A is a front view of another collar of the inventive subject matter in a collapsed configuration including an adjustment mechanism having first and second slots or notched rails. It should be appreciated that where the adjustment mechanism comprises a slot, the slot could be sized and dimensioned to receive one or more projections (e.g., a stem portion via a snap fit, etc.). It should also be appreciated that where an adjustment mechanism comprises a notched rail (recess), the rail, including the notched portions, could be sized and dimensioned to receive at least a tip of a projection.

As illustrated, collar 300 comprises a first slot and a second slot, each comprising an inner end (325, 305, respectively) and an outer end (335, 315, respectively). Where a projection of an adjustment mechanism is coupled with or snapped through a slot and at a resting position, it is contemplated that the projection could be biased towards an outer end of the slot, which can optionally be curved or hooked in any suitable direction to advantageously lock the projection in place, especially where the projection is additionally or alternatively biased towards an upper or lower portion of the slot.

FIG. 3B is a front view of the collar of FIG. 3A in an expanded configuration (e.g., projections are at fourth level 340). Collar 300 comprises first and second slots having a first level 310, a second level 320, a third level 330 and a fourth level 340. To adjust a position of a projection from one level (e.g., the first level, etc.) to another level (e.g., the second, third, fourth level, etc.), a user could squeeze first and second adjustment members (not shown) towards one another, and slide the projection to a desired level. Where the projections are biased towards outer ends of the slots, releasing the first and second adjustment members could cause the projections to automatically lock in place at the desired level.

FIG. 3C is a perspective view of the collar of FIGS. 3A and 3B in an expanded configuration. As illustrated, the projections of collar 300′s adjustment mechanism are locked in place on a fourth level 340 (See FIG. 3B). As the chin support and collar body are pivotally attached to one another via first pivot point 350 and second pivot point 360, and slidably attached to one another via protrusions and slots, adjustment of the projections from a lower-most positioned level to a higher-most positioned level will adjust a position of a chin piece relative to the collar body along a length of adjustability 370.

In some embodiments, it is contemplated that an adjustment mechanism of the inventive subject matter could have a resiliency that helps lock the components in place. Additionally or alternatively, a level of the adjustment mechanism (e.g., fourth level 340) could be further curved (e.g., c-shaped) to help lock a projection in place at the level.

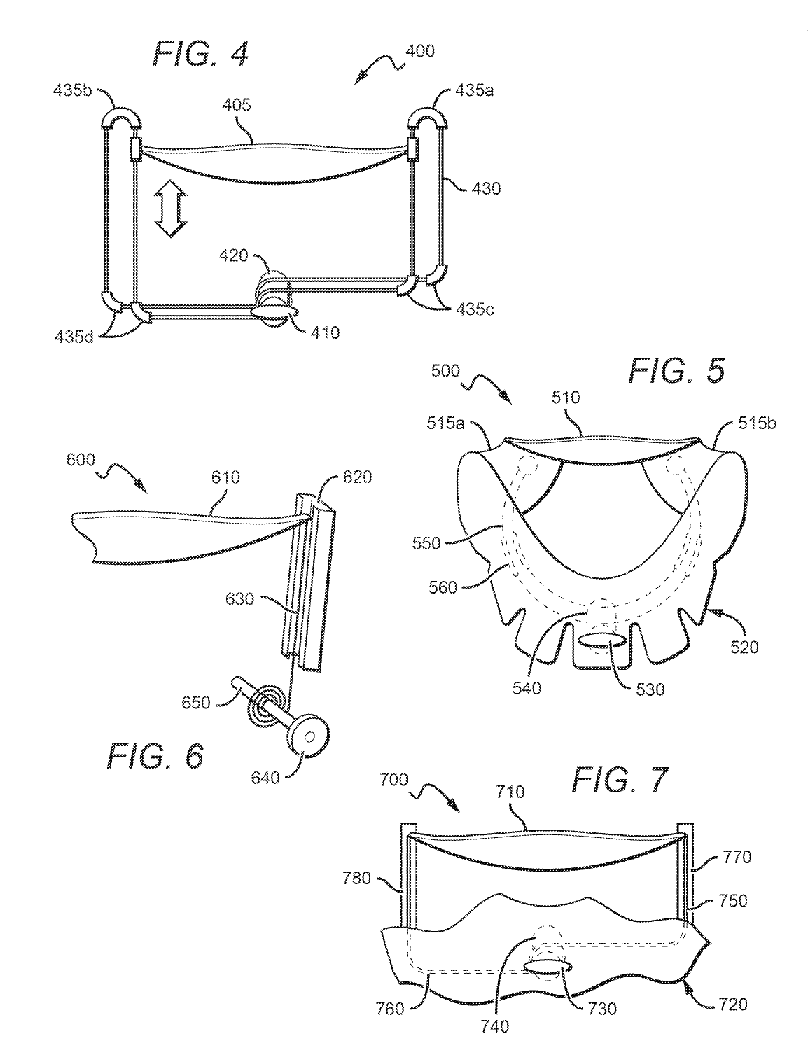

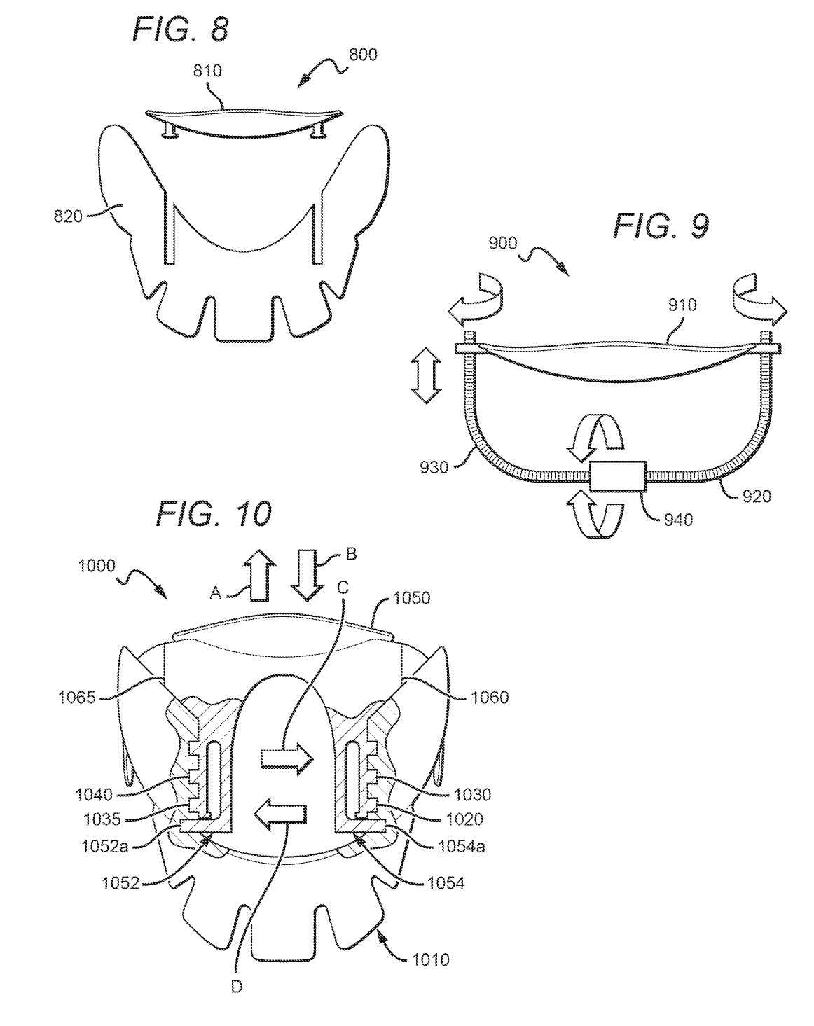

FIG. 4 illustrates an alternative adjustable collar 400 of the inventive subject matter comprising a closed loop line 430 that is coupled to each of a chin piece 405 and a collar body (not shown). Such an embodiment could be used, for example, where a chin piece 405 is directly coupled with a collar body. One way in which a chin piece 405 could be directly coupled with a collar body is by including protrusions on the chin piece 405 that pass through and slide within or through slots of the collar body (See e.g., FIG. 8). In alternative embodiments, the chin piece could be pivotally coupled to the collar body, such as described in U.S. Pat. No. 7,674,234 to Calco et al.

For example, the collar body could include or be coupled to a rotatable member comprising a knob 410 and a spool 420, and the line 430 could mate with the spool 420 such that a rotation of a knob 410 in a first direction causes the line 430 to wind around the spool in one direction while being “payed out” in the opposite direction, while a rotation of the knob 410 in a second direction causes the line to wind onto the spool 420, and be “payed out” from the spool 420 in the opposite direction. The line 430 could also pass through one or more slots of the chin piece 405, or be otherwise coupled to the chin piece 405 such that the unwinding or winding of the line on the spool 420 adjusts a position of the chin piece 405 relative to the collar body.

The adjustment mechanism could further comprise a first line guide 435a that guides the line 430 to a first portion of the chin piece 405 (e.g., a right side), and a second line guide 435b that guides the line 430 to a second portion of the chin piece 405 (e.g., a left side). Additionally or alternatively, the adjustment mechanism could comprise a third line guide 435c and a fourth line guide 435d. Such line guides could comprise, among other things, tabs secured to a surface of a device component having holes through which one or more lines could pass, and could be disposed on any suitable component of adjustable collar 400, including for example, the chin piece, the collar body, or a combination thereof

One should appreciate that the line could be coupled to the rotatable member in any suitable manner. For example, the line 430 could pass through a through-hole of the spool 420, and be configured to loop around the spool one or more times as it winds or unwinds.

In some embodiments, the adjustment mechanism could further include one or more springs or other mechanism configured to bias the chin piece at a first position relative to the collar body. A spring could bias the chin piece at a maximum or minimum vertical height relative to the collar body. For example, the collar could include one or more springs configured to bias the chin piece at a maximum vertical position relative to the collar body. In such embodiments, the adjustment mechanism can then be used to adjust the chin piece downward and thereby compress the one or more springs, or allow upward adjustment of the chin piece by allowing the spring to expand and release some of its potential energy.

Additionally or alternatively, a locking mechanism could be provided that prevents an inadvertent rotation of at least one of the spool and the knob once a desired chin piece—collar body arrangement is achieved. An exemplary locking mechanism could prevent a rotation of the spool and knob when the knob is in a fully inward configuration while allowing a rotation when the knob is in an outward position to prevent an increase in the line length. Still further, it is contemplated that a locking mechanism could be included in the slots of FIG. 8 to prevent a sliding of the protrusions relative to the slots.

It should be appreciated that a “line” could include a cord, rope, wire, string, strip, strap, thread, cable, rube, or a ligature, and could comprise any suitable material or materials, including for example, a nylon, a metal, a leather, a cotton, a plastic, an aerogel, an aluminum, a polyethylene, a polypropylene, or any other suitable material.

FIG. 5 illustrates another embodiment of an adjustable collar 500 of the inventive subject matter including a chin piece 510 coupled to a collar body 520 via first and second chin support members 515a and 515b, respectively. Preferably, the chin support members 515a and 515b allow for movement of the chin piece 510 relative to the collar body 520. For example, the chin support members 515a and 515b could have pivot points where they connect with one or both of the collar body 520 and chin piece 510. Additionally or alternatively, it is contemplated that the chin support members 515a and 515b could be slidably coupled with one or both of the collar body 520 and chin piece 510.

The line guides 560 shown are placed on the chin support members 515a, 515b and collar body 520, and configured to guide the line 550 from the spool 540 to the first and second chin support members 515a and 515b, respectively. As a user rotates the knob 530 located on the collar body 520, the line 550 can be configured to wind or unwind around the spool 540 to effectively adjust a vertical position (e.g., an exposed height) of the first and second chin support members 515a and 515b. The adjustment in exposed height of the support members could, in effect, adjust a vertical position of the chin piece 510 relative to the collar body 520.

As used herein, the term “exposed height” means a length of the portion of the support member that extends between the chin piece and the collar body. A length could be measured at, among other things, an outer edge of the support member, an inner edge of the support member, or a vertical or horizontal mid-point of the support member.

FIG. 6 illustrates an alternative embodiment of an adjustment mechanism 600 of the inventive subject matter comprising a first line 630 that is configured to wind around a first spool 650 and straighten into a first slot or rail 620. Preferably, the first line 630 is composed of a flexible but inelastic material. A portion of the first line 630 (here, the top portion) could be coupled to a chin piece 610 such that a turn of the knob 640 in a first direction (here, counter-clockwise) causes the chin piece 610 to increase its vertical position relative to the collar body to which the first knob is coupled. Viewed from another perspective, a turn of the knob in a second direction (here, clockwise) could cause the chin piece to move close to the collar body.

It should be appreciated that in some embodiments the first line could be coupled to a mid-portion of the chin piece such that a single adjustment mechanism could be used to adjust the entire chin piece relative to the collar body. Additionally or alternatively the first line could be coupled to a side portion of the chin piece, and a second inverse adjustment mechanism could be provided wherein the second mechanism's second line is coupled to an opposing side portion of the chin piece. In such embodiments two or more adjustment mechanisms (e.g., the adjustment mechanism of FIG. 6 and an inverse mechanism for the other side of the chin piece) could be used to adjust a chin piece relative to the collar body.

FIG. 7 shows a portion of an alternative adjustable collar 700 comprising a collar body 720 coupled to a chin piece 710 via an adjustment mechanism of the inventive subject matter. The adjustment member comprises a rotation member (spool 740, knob 730, etc.) that extends through a surface of the collar body 720 such that the knob 730 is readily accessible by a user, but the spool 740 is partially or entirely hidden from the user's view. The adjustment member further comprises first and second slots or rails 770 and 780, respectively, configured to receive first and second lines 750 and 760, respectively, made of a flexible but inelastic material. Each line is made from a material that can wind around a spool and straighten into a slot. The material is preferably of sufficient sturdiness or strength to adjust a position of at least a portion of the chin piece relative to the collar body when straightened into its respective slot. The first and second lines 750 and 760 could be wound around the first spool in an over-under configuration as shown such that a turn of the knob 730 in a first direction causes each of the lines to straighten further into its respective slots. This advantageously allows for simultaneous adjustment of both sides of the chin piece 710 via a single adjustment mechanism, thus simplifying use of the collar by a user.

FIG. 8 illustrates an alternative adjustable collar 800 of the inventive subject matter comprising a chin piece 810 that could be directly coupled with a collar body 820. As shown, the chin piece 810 includes protrusions that are sized and dimensioned to pass through and slide within or through slots (e.g., closed slots or open slots) of the collar body 820. It should be appreciated that there are various other contemplated ways in which a chin piece could be directly coupled with a collar body or other component. Furthermore, it is contemplated that a chin piece could be indirectly coupled to a collar body via another component (e.g., chin support member, etc.) in any suitable manner.

An alternative and novel embodiment of an adjustment mechanism is shown in FIG. 9. The adjustment mechanism 900 includes first and second flexible rods 920 and 930, respectively, which in the embodiment shown, extends from opposite ends of a chin piece 910 and is coupled with a rotatable member 940. The flexible rods could include male threads cut in opposite directions from the rotatable member, while the rotatable member could comprise a tube-like member that includes complementary female threads cut in opposite directions from a mid-point of the rotatable member. The rotatable member could be rotated in a first direction to cause each of the flexible rods to move towards a mid-portion of the rotatable member (thereby shortening a distance between the chin piece and collar body), or rotated in a second direction to cause each of the flexible rods to move towards a respective side of the rotatable member (thereby lengthening a distance between the chin piece and collar body).

It is contemplated that where an adjustment of up to n inches between two components is desirable, the rotatable member could have a threaded portion having a length of at least 2n. For example, where it is desirable for one component (e.g., chin piece, etc.) to be adjustable relative to a second component (e.g., collar body) by two inches, the rotatable member could be at least five inches in length, having two inches of internal threading at a first direction, two inches of internal threading at a second direction, and an unthreaded quarter inch on each side.

Preferably, the normal pressure applied by a user's chin when worn will not cause an adjustment in the position of the chin piece relative to the collar body. However, a locking mechanism could be included as necessary or desired to ensure that the user does not experience any unintentional adjustments.

FIG. 10 illustrates an alternative adjustable collar 1000 of the inventive subject matter. Collar 1000 includes a chin support 1050 having first and second side portions (1060 and 1065, respectively) and is adjustably coupled to the collar body 1010. Collar body 1010 optionally includes first and second rails (on internal side not shown) that are sized and dimensioned to receive side portions 1060 and 1065 of chin support 1050. It should be appreciated that the collar and chin support could alternatively or additionally be coupled in any other suitable manner, including for example, via at least one adjustment member and corresponding notch(es).

The collar body 1010 comprises a first set of notches (e.g., on a notched rail) including first notch 1020 and second notch 1030. Collar body 1010 further comprises a second set of notches including third notch 1035 and fourth notch 1040. Preferably, the sets each have at least two notches; however, it should be appreciated that a set of notches could comprise any suitable number of notches spaced apart by any suitable distance(s) to accommodate a desired adjustment.

In the embodiment shown in FIG. 10, chin support 1050 comprises a first adjustment member 1054 and second adjustment member 1052. The chin support 1050 and first and second adjustment members 1054 and 1052, respectively, can advantageously be made from a single piece of material (e.g., a single piece of injection molded material, etc.) for ease of manufacture, storage and use. However, it should also be appreciated that in some embodiments the chin support, first adjustment member and second adjustment member could each be made from separate pieces of material coupled with one another. Alternatively or additionally, the first and second adjustment members could compose a single piece of material that is coupled with the chin support. It should be appreciated that regardless of the number of pieces used, the adjustment members of some embodiments could be integral with the chin support.

First adjustment member 1054 comprises a first end 1054a, which is biased in direction C for placement in at least one of first notch 1020 and second notch 1030 in a resting position. Second adjustment member 1052 comprises a second end 1052a, which is biased in direction D for placement in at least one of third notch 1035 and fourth notch 1040. To adjust a height of the chin support 1050 relative to the collar body 1010, a user can squeeze first and second adjustment members towards one another (i.e., first adjustment member 1054 towards direction D and second adjustment member 1052 towards direction C) and slide chin support 1050 upwards towards direction A or downwards towards direction B. Upon release at a desired position, the adjustment members could advantageously lock the chin support 1050 in place.

A space between first and second adjustment members 1054, 1052 can comprise any suitable distance when in a locked position (e.g., when ends of each adjustment members remain within at least one notch in a resting position, etc.). In some preferred embodiments, especially in those that allow a user to adjust a collar using a single hand, the distance can be less than or equal to 6 inches, less than or equal to 5 inches, less than or equal to 4 inches, less than or equal to 3 inches, or even less than or equal to 2 inches. Additionally or alternatively, the adjustment members could each comprise curved or looped shapes (e.g., finger stops, etc.) that are sized and dimensioned to receive a user's finger. Such configurations allow a user to, among other things, use a thumb and index finger of a single hand to easily adjust a collar of the inventive subject matter.

The adjustment members, chin support and collar body could each be made of any suitable material or materials, including for example, a plastic, a cotton, a polyester, a foam, a metal, a wire, a rubber, a polysterene, a polyvinyl chloride, a nylon, or any other suitable material. In some preferred embodiments, the adjustment members could be made of a material that is sized and dimensioned to be sufficiently flexible to allow a wearer to reduce a distance between them by at least 0.5 cm, at least lcm, at least 2 cm, or even at least 3 cm or more to thereby release the adjustment members from a locked position. Additionally or alternatively, the adjustment members could be made of a material that is sized and dimensioned to be sufficiently rigid such that the adjustment members could remain in a locked position when the adjustable collar is worn by the wearer.

The inventive subject matter also includes methods of providing an adjustable collar to a user, and instructing the user to adjust a height of a chin support relative to the collar body. In some aspects, an adjustable collar as described above could be provided to a user with oral, written or other form of instructions. The instructions could instruct or guide the user to, for example, move first and second adjustable members (e.g., retractable portions, etc.) of a chin support towards one another, guide the chin support and adjustable members vertically relative to the collar body, and release the adjustable members at a desired height relative to the collar body.

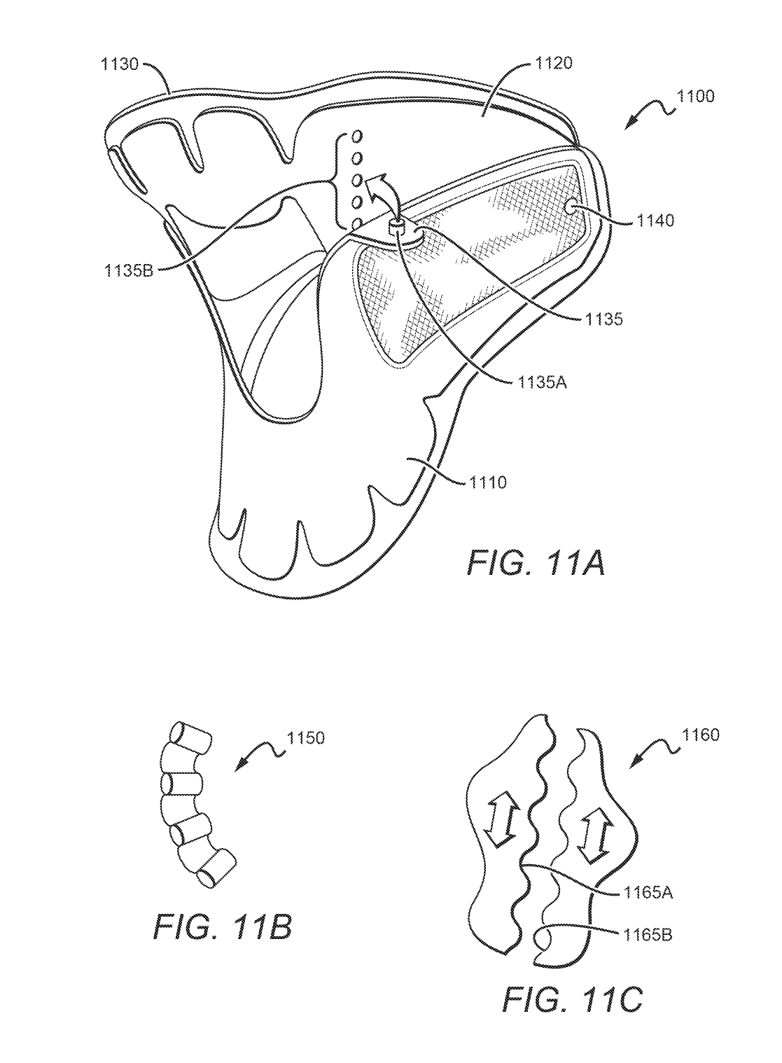

FIG. 11A illustrates yet another embodiment of an adjustable collar of the inventive subject matter. Adjustable collar 1100 includes a collar body 1110 and chin support 1120 coupled at an attachment point 1140. Chin support 1120 comprises or is coupled to chin piece 1130, and includes a first plurality of openings (through-holes) or recesses (not through-holes) that are sized and dimensioned to receive at least a portion of a first protrusion 1135A. First protrusion 1135A is coupled to or a part of first collar tab 1135, which is a bendable tab that is coupled to or a part of collar body 1110. First protrusion 1135A can be inserted and removed from one or more openings of the first plurality of openings 1135B to adjust a position (e.g., a vertical position) of chin piece 1130 relative to collar body 1110.

FIG. 11B illustrates an optional first portion 1150, which can be included on a surface of the adjustable collar 1100, and include a plurality of bumps or ridges, or otherwise nonflat portions. First portion (e.g., 1150) could abut a second portion of the collar 1100 (one portion being on an outer surface of at least one of the chin support 1120 and collar body 1110, the other portion being on an inner surface of at least one of the chin support 1120 and collar body 1110), and could cooperate to prevent or thwart a sliding of the chin support 1120 relative to the collar body 1110. FIG. 11C illustrates interlocking portions 1160, which can be included on adjustable collar 1100. For example, first portion 1165A could be included on an outer surface of a chin piece 1130 and configured to abut second portion 1165B, included on an inner surface of a collar body 1110. Preferably, the bumps or ridges of each of first portion 1165A and second portion 1165B are placed, sized and dimensioned such that first protrusion 1135A will align with some or all of the first plurality of openings 1135B when first portion 1165A and second portion 1165B interlock.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. An adjustable collar comprising:
a chin piece coupled to a collar body;
an adjustment mechanism coupled to the chin piece that allows for an adjustment of the chin piece relative to the collar body;
wherein the adjustment mechanism comprises:
first and second movable adjustment members including first and second projections, respectively; and
first and second rails disposed on the collar body; and
wherein the first rail comprises a first plurality of level, each sized and dimensioned to receive a stem portion of the first projection, and wherein the second rail comprises a second plurality of levels, each sized and dimensioned to receive a stem portion of the second projection.

2. The adjustable collar of claim 1, wherein the chin piece is coupled to the collar body via the adjustment mechanism.

3. The adjustable collar of claim 1, further comprising a chin support that is pivotally coupled with the collar body.

4. The adjustable collar of claim 3, wherein the chin support is coupled with the collar body such that the chin support is positioned behind the collar body.

5. The adjustable collar of claim 3, wherein the first projection composes a left side of the chin support, wherein the second projection composes a right side of the chin support, wherein the first rail is disposed on a left side of the collar body, and wherein the second rail is disposed on a right side of the collar body.

6. The adjustable collar of claim 1, wherein the first and second adjustment members are biased towards a locked position wherein the first projection sits on an outer end of a level of the first plurality of levels, and the second projection sits on an outer end of a level of the second plurality of levels.

7. The adjustable collar of claim 1, wherein a distance between an inner end of the first rail and an inner end of the second rail is less than or equal to five inches.

8. The adjustable collar of claim 1, wherein at least one of the first and second adjustment members comprises a finger block.

9. The adjustable collar of claim 1, wherein the first and second adjustment members are sufficiently flexible to allow the wearer to reduce a distance between the first and second adjustment members by at least 1 cm.

10. The adjustable collar of claim 1, wherein the first and second adjustment members are sufficiently flexible to allow the wearer to reduce a distance between the first and second adjustment members by at least 2 cm.

11. An adjustable collar comprising:
a chin piece coupled to a collar body via a chin support; and
an adjustment mechanism comprising (i) first and second retractable members disposed on the chin support and including or coupled with first and second protrusions, respectively, and (ii) first and second notched rails disposed on the collar body, each notched rail including a plurality of levels; and
wherein the adjustment mechanism is configured to allow the wearer to adjust a height of the chin piece relative to the collar body by retracting the first and second retractable members.

12. The adjustable collar of claim 11, wherein the first and second retractable members are substantially horizontally aligned when the adjustable collar is worn by the wearer.

13. The adjustable collar of claim 11, wherein the first and second notched rails are substantially horizontally aligned when the adjustable collar is worn by the wearer.

14. The adjustable collar of claim 11, wherein the chin support comprises the chin piece.

15. The adjustable collar of claim 11, wherein the first and second retractable members are biased towards a locked position wherein the first projection sits on a outer end of a level of the plurality of levels of the first notched rail, and the second projection sits on an outer end of a level of the plurality of levels of the second notched rail.

16. The adjustable collar of claim 11, wherein at least one of the first and second retractable members comprises a finger block.

17. The adjustable collar of claim 11, wherein the first and second retractable members are sufficiently flexible to allow the wearer to reduce a distance between the first and second retractable members by at least 1 cm.

18. The adjustable collar of claim 11, wherein the first and second retractable members are sufficiently flexible to allow the wearer to reduce a distance between the first and second retractable members by at least 2 cm.
